(19) United States
(12) Patent Application Publication
(10) Pub. No.: US 2025/0161378 A1
(43) Pub. Date: May 22, 2025

(54) COMPOSITE BACTERIAL AGENT AND APPLICATION THEREOF

(71) Applicant: HEALTH AND HAPPINESS (H&H) HONG KONG LIMITED, Hong Kong (CN)

(72) Inventors: Hua ZHANG, Hong Kong (CN); Lingling ZHAO, Hong Kong (CN); Ying LI, Hong Kong (CN)

(73) Assignee: HEALTH AND HAPPINESS (H&H) HONG KONG LIMITED, Hong Kong (CN)

(21) Appl. No.: 19/028,581

(22) Filed: Jan. 17, 2025

Related U.S. Application Data
(63) Continuation of application No. PCT/CN2023/128889, filed on Oct. 31, 2023.

(30) Foreign Application Priority Data
Oct. 17, 2023 (CN) 202311342233.5

(51) Int. Cl. A61K 35/745; A61P 1/04; C12N 1/20; C12R 1/225; C12R 1/46

(52) U.S. Cl. CPC A61K 35/745; A61P 1/04; C12N 1/20; C12R 2001/225; C12R 2001/46

(57) ABSTRACT

A composite bacterial agent, and an application thereof. The composite bacterial agent includes Bifidobacterium longum, Enterococcus faecalis, Enterococcus faecium, Ligilactobacillus animalis, and Companilactobacillus muruki, and provides significant effect in treating intestinal disorders and inflammatory bowel disease in felines caused by bacteria.

### CROSS-REFERENCE TO RELATED APPLICATIONS

The present application is a Bypass Continuation application of PCT Patent Application No. PCT/CN2023/128889, filed on Oct. 31, 2023, which claims priority to Chinese Patent Application No. 202311342233.5, filed on Oct. 17, 2023. The content of all aforesaid applications is incorporated herein by reference.

### FIELD

The present invention relates to the field of microorganisms, in particular to a composite bacterial agent and an application thereof.

### BACKGROUND

Felines are one of the primary companion animals for humans. With the improvement in people's quality of life, there has been a growing emphasis on feline health and well-being.

One of the most common health issues in felines is intestinal disease, often caused by intestinal homeostasis imbalance. Such imbalance is typically associated with gut microbiota dysbiosis and inflammatory bowel disease (IBD). Currently, the primary treatment for bacterial-induced intestinal disorders and IBD in felines involves administering antibiotics. However, antibiotics not only eliminate harmful bacteria in the gut but also kill beneficial bacteria, which can prolong the treatment and recovery period in certain cases. Additionally, excessive or long-term use of antibiotics may increase bacterial resistance within the same individual, leading to diminished treatment efficacy over time. At present, there are no widely available alternative medications with superior therapeutic effectiveness.

Therefore, the prior art still needs improvement.

### SUMMARY

In view of the aforementioned deficiencies in the prior art, the objective of the present invention is to provide a composite bacterial agent, with the aim of improving the therapeutic efficacy for intestinal disorders and inflammatory bowel disease in felines.

In order to achieve the above objective, the present disclosure adopts the following technical solutions:

A composite bacterial agent comprises Bifidobacterium longum, Enterococcus faecalis, Enterococcus faecium, Ligilactobacillus animalis, and Companilactobacillus muruki.

An application of the composite bacterial agent as described above in a preparation of a drug for treating intestinal disorders in felines.

An application of the composite bacterial agent as described above in a preparation of a drug for treating inflammatory bowel disease in felines.

### Beneficial Effects

The present invention provides a composite bacterial agent comprising Bifidobacterium longum, Enterococcus faecalis, Enterococcus faecium, Ligilactobacillus animalis, and Companilactobacillus muruki. Through the synergistic effect of these five bacterial strains, the composite bacterial agent can effectively treat intestinal disorders and inflammatory bowel disease in felines.

### BRIEF DESCRIPTION OF THE DRAWINGS

FIG. 1 shows the fecal score chart.

FIG. 2 shows an IgA content in the blood of felines in each group after 30 days of treatment with the drug or composite bacterial agent.

FIG. 3 shows an IgG content in a blood of felines in each group after 30 days of treatment with the drug or composite bacterial agent.

FIG. 4 shows an IgM content in a blood of felines in each group after 30 days of treatment with the drug or composite bacterial agent.

FIG. 5 shows the fecal scoring criteria.

### DETAILED DESCRIPTION OF EMBODIMENTS

The present invention provides a composite bacterial agent and the application thereof. To make the objective, technical solution, and effect of the present invention clearer and more explicit, the following detailed description is provided with reference to the accompanying drawings and embodiments. It should be understood that the embodiments described herein are only intended to explain the present invention and are not intended to limit the present invention.

It should be noted that in the following embodiments, Companilactobacillus muruki HHP001, which is the same as Companilactobacillus muruki A-148 with the deposit number GDMCC No. 62868, was deposited on Oct. 13, 2022, at the Guangdong Microbial Culture Collection Center (GDMCC);

Ligilactobacillus animalis HHP002, which is the same as Ligilactobacillus animalis A-11-15 with the deposit number GDMCC No. 62867, was deposited on Oct. 13, 2022, at the Guangdong Microbial Culture Collection Center (GDMCC);

Enterococcus faecalis HHP003, which is the same as Enterococcus faecalis A-A-2 with the deposit number GDMCC No. 62869, was deposited on Oct. 13, 2022, at the Guangdong Microbial Culture Collection Center (GDMCC);

Bifidobacterium longum HHP004, which is the same as Bifidobacterium longum A-1-12 with the deposit number GDMCC No. 62866, was deposited on May 9, 2023, at the Guangdong Microbial Culture Collection Center (GDMCC);

Enterococcus faecium HHP005, which is the same as Enterococcus faecium M-3 with the deposit number GDMCC No. 62870, was deposited on Oct. 13, 2022, at the Guangdong Microbial Culture Collection Center (GDMCC).

### Embodiment

### 1.1 Animal Experiments

The felines were randomly divided into five groups, with six felines in each group, the groups were as follows:
Control group (Group N): no additional treatment;
Drug group (Group A): From day 1 to day 8, the felines were continuously challenged by administering E. coli powder capsules once daily, with a dose of 1×10⁹ CFU per kilogram of body weight; from day 9 to day 30, the felines were given amoxicillin-clavulanate potassium, once daily, at a dose of 50 mg (1 tablet) each time;
Single strain probiotic group (Group 1S): From day 1 to day 8, the felines were continuously challenged in the same manner as the drug group; from day 9 to day 30, the felines were given Enterococcus faecalis HHP003, with a dose of 1×10⁹ CFU per kilogram of body weight;
Three strains probiotic group (Group 3S): From day 1 to day 8, the felines were continuously challenged in the same manner as the drug group; from day 9 to day 30, the felines were given a combination of Enterococcus faecalis HHP003, Bifidobacterium longum HHP004, and Enterococcus faecium HHP005, with a dose of 1×10⁹ CFU per kilogram of body weight for each strain;
Five strains probiotic group (Group 5S): From day 1 to day 8, the felines were continuously challenged in the same manner as the drug group; from day 9 to day 30, the felines were given a combination of Companilactobacillus muruki HHP001, Ligilactobacillus animalis HHP002, Enterococcus faecalis HHP003, Bifidobacterium longum HHP004, and Enterococcus faecium HHP005, with a dose of 1×10⁹ CFU per kilogram of body weight for each strain;
The felines were bled on day 0, day 8, and day 30 to measure the relevant blood indicators.

The feces of the felines were scored, and the fecal scoring criteria were based on the following reference:

Evaluation of canned therapeutic diets for the management of felines with naturally occurring chronic diarrhea, Journal of Feline drug and Surgery, 14 (10) 669-677, DOI: 10.1177/1098612X12446906.

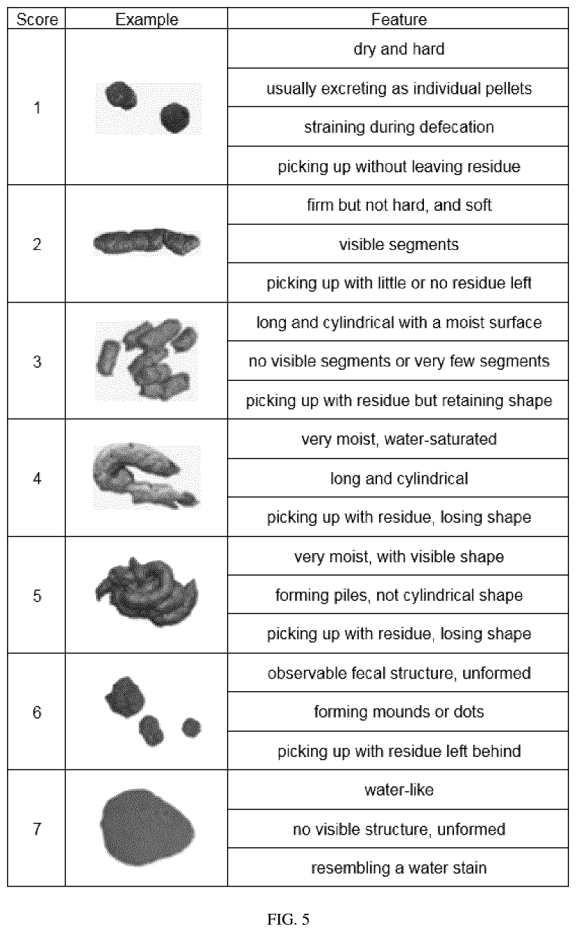

The fecal scoring criteria is shown in FIG. 5. In the scoring criteria of FIG. 5, the closer the score is to 2 points, the better the health status of the feline is.

### 1.2 the Results of the Fecal Tests are as Follows

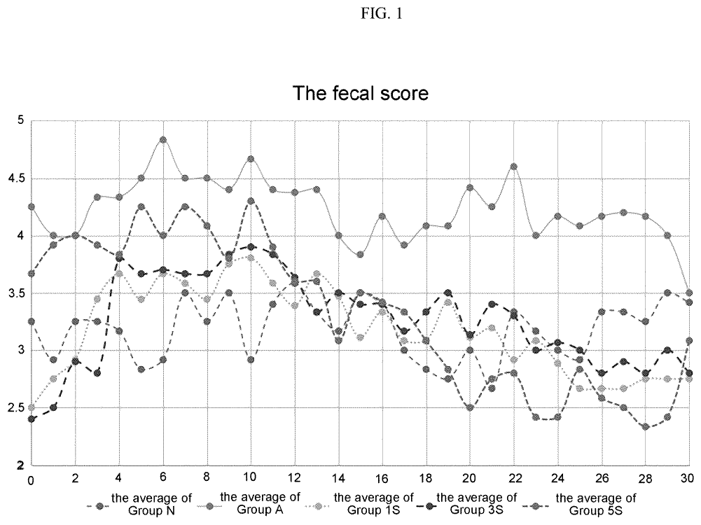

Refer to FIG. 1, which shows the daily trend of average fecal scores for each group of felines. For the Control group (Group N), which does not undergo any challenge treatment and is not given probiotics or antibiotics, the fecal scores of Group N in FIG. 1 remain stable between 2.5 and 3.5.

The fecal scores of the Drug group (Group A) gradually increase after the challenge treatment. After discontinuing the challenge and administering antibiotics, there is a significant downward trend in fecal scores from day 10 to day 15. However, there is a relapse on days 20 and 22, indicating that while the use of antibiotics can help to repair intestinal homeostasis in the short term, long-term use may lead to the development of bacterial resistance.

The fecal scores of the Single strain probiotic group (Group 1S) also gradually increase after the challenge treatment, and the increase is significantly greater than that of the drug group (Group A), indicating that the felines in this group are more sensitive to the challenge. After discontinuing the challenge and administering Enterococcus faecalis HHP003, the fecal scores of Group 1S continuously decrease from day 10 to day 25. Additionally, during the same period, the rate of decrease in fecal scores for Group 1S is significantly faster than that of Group A, suggesting that Enterococcus faecalis HHP003 is more effective in treating intestinal disorders in felines compared to the drug group (Group A).

The fecal scores of the Three strains probiotic group (Group 3S) also gradually increase after the challenge treatment. After discontinuing the challenge and administering Enterococcus faecalis HHP003, Bifidobacterium longum HHP004, and Enterococcus faecium HHP005, the trend in fecal scores is similar to that of the single probiotic group (Group 1S). This indicates that the combined use of Enterococcus faecalis HHP003, Bifidobacterium longum HHP004, and Enterococcus faecium HHP005 is more effective in treating intestinal disorders in felines compared to the drug group (Group A).

The fecal scores of the Five strains probiotic group (Group 5S) also gradually increase after the challenge treatment, and the increase is similar to that of the drug group (Group A). After discontinuing the challenge and administering Companilactobacillus muruki HHP001, Ligilactobacillus animalis HHP002, Enterococcus faecalis HHP003, Bifidobacterium longum HHP004, and Enterococcus faecium HHP005, the fecal scores decrease significantly from day 10 to day 20, and the decrease rate of the fecal scores is significantly faster than the other three groups. The fecal scores of Group 5S in the end is lower than that of the Group 1S and the Group 3S, which indicates that the combined use of Companilactobacillus muruki, Ligilactobacillus animalis, Enterococcus faecalis, Bifidobacterium longum, and Enterococcus faecium provides a better treatment and shorter treatment duration.

### 1.3 IgA Content in the Blood of Felines on Day 30 after Feeding with Drugs or Probiotics

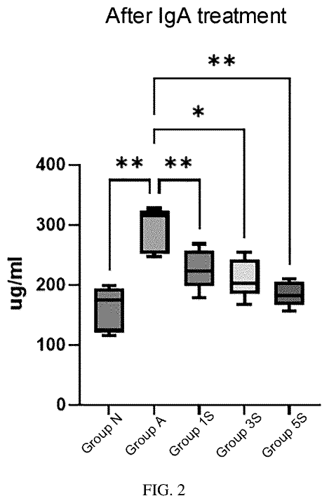

Refer to FIG. 2, which shows the IgA content in the blood of each group of felines on day 30. As shown in FIG. 2, it can be seen that the Control group (Group N), which did not undergo any challenge treatment or receive drugs or probiotics, has IgA levels within the normal range.

The IgA content in the Drug group (Group A) is very high, and there is a significant difference compared to the Control group (Group N), indicating that although the Drug group (Group A) solves the intestinal disorders in the felines, the inflammation caused by the challenge is not effectively eliminated.

The IgA contents in the Single strain probiotic group (Group 1S) and the Three strains probiotic group (Group 3S) are lower than that in the Drug group (Group A), and there is no significant difference compared to the Control group (Group N), indicating that the Single strain probiotic group (Group 1S) and the Three strains probiotic group (Group 3S) can effectively eliminate the inflammation caused by the challenge and treat inflammatory bowel disease, but a higher content is observed compared to the Five strains probiotic group (Group 5S).

The IgA content in the Five strains probiotic group (Group 5S) is lower than that in the Drug group (Group A), and there is no significant difference compared to the Control group (Group N), indicating that the combination of Companilactobacillus muruki HHP001, Ligilactobacillus animalis HHP002, Enterococcus faecalis HHP003, Bifidobacterium longum HHP004, and Enterococcus faecium HHP005 can also effectively eliminate the inflammation caused by the challenge and treat inflammatory bowel disease. In addition, the IgA content in the Group 5S is lower than that of the Group 1S and the Group 3S, and equal to that of the Group N.

### 1.4 IgG Content in the Blood of Felines on Day 30 after Feeding with Drugs or Probiotics

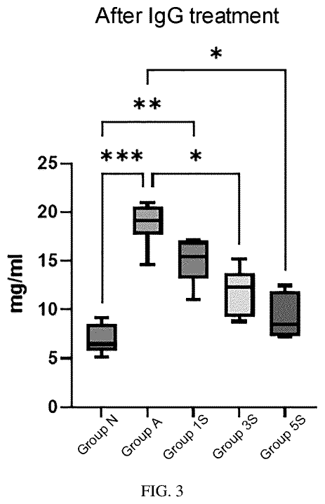

Refer to FIG. 3, which shows the IgG content in the blood of each group of felines on day 30. As shown in FIG. 3, it can be seen that the Control group (Group N), which did not undergo any challenge treatment or receive drugs or probiotics, has IgG levels within the normal range.

The IgG content in the Drug group (Group A) is very high, and there is an extremely significant difference compared to the Control group (Group N), indicating that the inflammatory issues in the felines of the Drug group (Group A) are still very evident, and the use of antibiotics could not effectively eliminate the inflammation caused by the challenge.

The IgG contents in the Single strain probiotic group (Group 1S) and the Three strains probiotic group (Group 3S) are lower than that in the Drug group (Group A), and there is no significant difference between the Single strain probiotic group (Group 1S) Drug group (Group A), but a significant difference between the Three strains probiotic group (Group 3S) and the Drug group (Group A), indicating that the Single strain probiotic group (Group 1S) and the Three strains probiotic group (Group 3S) can, to some extent, cure the inflammation caused by the challenge and treat inflammatory bowel disease.

The IgG content in the Five strains probiotic group (Group 5S) is significantly different from that in the Drug group (Group A), lower than that in the Single strain probiotic group (Group 1S) and the Three strains probiotic group (Group 3S), and shows no significant difference compared to the Control group (Group N), indicating that, compared to the other three groups of drug or bacterial agent, the combination of Companilactobacillus muruki HHP001, Ligilactobacillus animalis HHP002, Enterococcus faecalis HHP003, Bifidobacterium longum HHP004, and Enterococcus faecium HHP005 provides the best effect in eliminating inflammation.

### 1.5 the IgM Content in the Blood of Felines on Day 30 after Feeding with Drugs or Probiotics

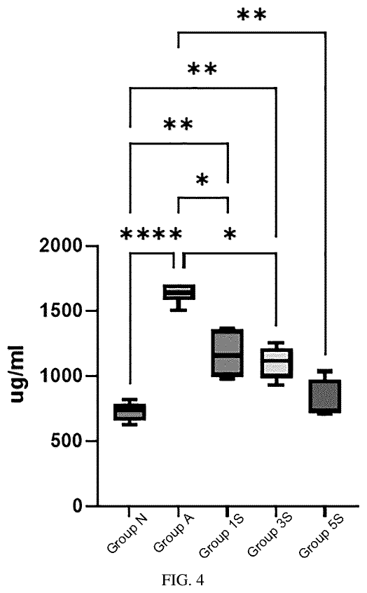

Refer to FIG. 4, which shows the IgM content in the blood of each group of felines on day 30. As shown in FIG. 4, it can be seen that the control group (Group N), which did not undergo any challenge treatment or receive drugs or probiotics, has IgM levels within the normal range.

The IgM content in the Drug group (Group A) is very high, and there is an extremely significant difference compared to the Control group (Group N), indicating that the inflammatory issues in the felines of the Drug group (Group A) are still very evident, and the use of antibiotics is not effective in treating the inflammation caused by the challenge.

The IgM content in the Single strain probiotic group (Group 1S) and the Three strains probiotic group (Group 3S) are lower than that in the Drug group (Group A), and there is a significant difference compared to the Drug group (Group A), indicating that the Single strain probiotic group (Group 1S) and the Three strains probiotic group (Group 3S) can cure the inflammation caused by the challenge and treat inflammatory bowel disease.

The IgM content in the Five strains probiotic group (Group 5S) shows a significant difference compared to the Drug group (Group A), and no significant difference compared to the control group (Group N), indicating that, compared to the other three groups of drug or bacterial agent, the combination of Companilactobacillus muruki HHP001, Ligilactobacillus animalis HHP002, Enterococcus faecalis HHP003, Bifidobacterium longum HHP004, and Enterococcus faecium HHP005 provides the best effect in eliminating inflammation, and the IgM content for felines is recovered to normal level at day 30.

In summary, while antibiotic treatment has shown some efficacy in treating feline intestinal disorders, it is associated with prolonged treatment periods, limited effectiveness, and an inability to effectively treat feline inflammatory bowel disease. In contrast, the probiotics in Group 1S, Group 3S, and Group 5S not only can treat feline intestinal disorders but also demonstrate significantly better therapeutic effects compared to the antibiotics (Group A), as reflected by fecal scores and immunoglobulin indicators. Furthermore, a comprehensive comparison of fecal scores and IgA, IgG, and IgM contents across Group 1S, Group 3S, and Group 5S reveal that the Group 5S exhibits the best therapeutic effect. The Group 5S is the first group that restores fecal scores to normal levels, and the time is even shorter than the control group (Group N), indicating further improvement in intestinal microecology. Additionally, IgA, IgG, and IgM levels in blood in Group 5S are the lowest and comparable to those in the control group (Group N) in day 30, which suggests, in general, that the combination of Bifidobacterium longum, Enterococcus faecalis, Enterococcus faecium, Ligilactobacillus animalis, and Companilactobacillus muruki demonstrates excellent therapeutic efficacy for inflammatory bowel disease, offering promising application prospects and significant practical value.

It is understood that for those skilled in the art, equivalent substitutions or modifications can be made based on the technical solutions and inventive concepts of the present disclosure, and all such modifications or substitutions should fall within the scope of protection of the appended claims of the present disclosure.

What is claimed is:

1. A composite bacterial agent, comprising Bifidobacterium longum, Enterococcus faecalis, Enterococcus faecium, Ligilactobacillus animalis, and Companilactobacillus muruki.

2. An application of the composite bacterial agent according to claim 1 in a preparation of a drug for treating intestinal disorders in felines.

3. An application of the composite bacterial agent according to claim 1 in a preparation of a drug for treating inflammatory bowel disease in felines.
